(19) United States
(12) Patent Application Publication
(10) Pub. No.: US 2026/0083966 A1
(43) Pub. Date: Mar. 26, 2026

(54) RESERVOIR FOR USE WITH A MEDICAL DEVICE AND SYSTEM FOR INTERVENTIONAL DRUG DELIVERY

(71) Applicant: CONTINUITY BIOSCIENCES, LLC, Cary, NC (US)

(72) Inventors: William Daunch, Cary, NC (US); Javier de Ana, Boise, ID (US); Casey Haigh, Cary, NC (US); Brendan Berkel, Raleigh, NC (US); Elliott Hannah, Raleigh, NC (US)

(21) Appl. No.: 19/339,069

(22) Filed: Sep. 24, 2025

Related U.S. Application Data
(60) Provisional application No. 63/698,954, filed on Sep. 25, 2024.

(51) Int. Cl. A61N 1/32; A61K 9/00; A61K 31/7105; A61M 31/00; A61N 1/05

(52) U.S. Cl. CPC A61N 1/325; A61K 9/0009; A61K 9/0024; A61K 31/7105; A61M 31/002; A61N 1/05; A61M 2205/04

(57) ABSTRACT

A surgically implantable reservoir is provided for implantation into a patient for use in an iontophoresis system. The reservoir and system are used for local drug delivery through a target site of internal or external body tissue. The iontophoresis system includes a source electrode and a counter electrode in electrical communication with the source electrode for forming a localized electric field at the target site. A fluid cargo may comprise a drug solution to be delivered to target tissue. For example, a chemotherapeutic drug solution or any other fluid polar substance may be delivered via the reservoir. In one embodiment, the fluid cargo may include ribonucleic acid molecules.

### CROSS-REFERENCES

This application claims the benefit of U.S. Provisional patent application No. 63/698,954 , filed Sep. 25, 2024, entitled Implantable Reservoir for Use with a Medical Device and System for Interventional Drug Delivery, the contents of which are incorporated herein in their entirety.

### BACKGROUND

A medical device and system for interventional drug delivery are described and, more particularly, a reservoir is described for use with an iontophoresis device and system for targeted drug delivery.

Delivery of chemotherapy directly into affected organs and other parts of the body offers a solution for cancers that are difficult to treat with systemic therapy alone. In one application, a device designed to infuse chemotherapy drugs is implanted directly into a tumor. This technology allows for more targeted drug delivery of higher doses directly to the tumor, largely sparing surrounding tissues. By treating the tumor directly, doctors can theoretically shrink the tumor to an operable size with a smaller dose of chemotherapy. This approach should also significantly reduce the side effects of systemic toxicity on the patient.

An example of a disease that is difficult to treat is pancreatic cancer. The pancreas is in a challenging location near critical organs and vessels. As a pancreatic tumor grows into adjacent tissues, it can invade the liver or the stomach, and more often invades local vasculature, rendering the tumor inoperable. Moreover, the pancreatic tumor is resistant to conventional systemic chemotherapy due to a dense fibroblastic stroma which surrounds the tumor. Current systemic treatments attempt to overcome these difficulties by increasing the dosage of intravenously administered chemotherapy. However, this rarely works, and the high dosage is exceptionally hard on the patient.

A medical device that implants directly onto the pancreas may be used to infuse chemotherapy drugs, such as gemcitabine, directly into a pancreatic tumor. The device uses iontophoresis to drive therapeutic chemotherapy drugs into the tumor using electrical currents that pass through the drug solution into the tissue. The device includes an implantable reservoir containing the drugs and an electrode. The reservoir is implanted on the pancreas and connected through the abdomen to an infusion pump and electrical leads from the electrode. The circuit is completed by a second electrode on the back of the patient for generating an electrical field. Iontophoresis uses electromotive and electro-osmotic forces which cause the chemotherapy drugs to pass across the stroma and into the tumor.

One such device is described in U.S. Pat. No. 10,695,562, titled Interventional Drug Delivery System and Associated Methods, the contents of which patent is hereby incorporated by reference herein in their entirety. A similar device, which is used in combination with therapeutic radiation, is described in U.S. Pat. Nos. 10,532,226 and 11,351,399, titled Combined Local Delivery of Therapeutic Agents Using Interventional Devices and Radiation, the contents of both of which patents are also hereby incorporated by reference herein in their entirety. An implantable reservoir is described in Applicant's co-pending U.S. patent application, titled Implantable Reservoir for Use with a Medical Device and System for Interventional Drug Delivery, having been published as U.S. Published Application No. 2022/0218978, the contents of which are also incorporated herein by reference herein in their entirety.

A problem with an iontophoresis device is the electrolysis that occurs at the surface of the electrode produces bubbles in the fluid in the reservoir. The bubbles adhere to the electrode surface causing the impedance of the electrode to change, which then requires a higher voltage for maintaining functionality. However, the voltage must remain below 25V to ensure there are no adverse effects on the patient. The bubbles in the reservoir also reduce the volume available for therapeutic fluid leading to diminished delivery results. In addition, the orientation of the device on the pancreas may affect any mechanism to sweep the bubbles off the electrode.

For the foregoing reasons, there is a need for an implantable reservoir for use with an iontophoresis device and system which reduces gas generated by electrolysis and minimizes adherence of bubbles to the electrode surface by removal of the bubbles formed. Ideally, the bubble removal process should work regardless of the orientation of the reservoir.

### SUMMARY

A surgically implantable reservoir is provided for implantation into a patient for use in an iontophoresis system. The reservoir and system are used for local drug delivery through a target site of internal or external body tissue. The iontophoresis system includes a source electrode and a counter electrode in electrical communication with the source electrode for forming a localized electric field at the target site. A fluid cargo comprises a drug solution to be delivered to the target tissue. For example, a chemotherapeutic drug solution or any other fluid polar substance may be delivered via the reservoir. In one embodiment, the fluid cargo may include ribonucleic acid molecules.

In one embodiment, a surgically implantable reservoir is provided for implantation into a patient for use in an iontophoresis system for local drug delivery through a target site of internal body tissue. The iontophoresis system includes a source electrode and a counter electrode in electrical communication with the source electrode for forming a localized electric field at the target site. The reservoir comprises a housing having an inner surface defining a first enclosed chamber having an inlet opening spaced from an outlet opening for flow of fluid including the drug through the first chamber from the inlet opening to the outlet opening. A second enclosed chamber has a smaller volume than the first chamber and an inlet opening spaced from an outlet opening for flow of fluid including the drug through the second chamber from the inlet opening to the outlet opening. The outlet of the first chamber is in fluid communication with the inlet of the second chamber, and the second chamber is adapted for holding the source electrode. The the housing comprises a first membrane at least partially defining the first chamber and capable of interacting with the localized electric field. The membrane allows drug to pass through the membrane and into the targeted tissue when the localized electric field is applied. A second second membrane separates the first chamber from the second chamber and allows drug to pass through the membrane and into the first chamber when the localized electric field is applied. Means are provided for securing the housing to the tissue of the target site. Fluid flow through the second chamber from the inlet opening to the outlet opening moves gas bubbles formed by electrolysis from the surface of the electrode and carries the bubbles through the outlet opening of the second chamber.

The outlet opening in the second chamber may be opposite the inlet opening of the second chamber.

The housing securing means comprises a skirt around at least a portion of the reservoir, wherein the skirt may be sutured to tissue at the target site. The skirt has suture openings. The housing securing means may comprise a biological adhesive, microneedles, or staples.

The membrane may comprise natural or synthetic polyomers, including cellulose acetate, polysulfone, polycarbonate, polyamide, and polyacryl-polyamide acrylate.

A reservoir is also provided for use in an iontophoresis system for local drug delivery through an external target site of body tissue. The iontophoresis system includes a source electrode and a counter electrode in electrical communication with the source electrode for forming a localized electric field at the target site. The reservoir comprises a housing for accommodating the source electrode. The housing has an inner surface defining an enclosed chamber having an inlet opening spaced from an outlet opening for flow of fluid including the drug through the chamber from the inlet opening to the outlet opening. A membrane at least partially defines the chamber and is capable of interacting with the localized electric field. The membrane allows drug to pass through the membrane and into the targeted tissue when the localized electric field is applied. An inlet tube is in fluid communication with the inlet of the housing, and an outlet tube is in fluid communication with the outlet of the housing and extends at an angle of between about 30 degrees and about 90 degrees relative to a plane including the membrane. A handle is secured to the housing for manual placement of the membrane against the tissue of the target site. Fluid flow through the chamber from the inlet opening to the outlet opening moves gas bubbles formed by electrolysis from the surface of the electrode and carries the bubbles through the outlet opening.

The outlet opening may be opposite the inlet opening. The membrane may comprise natural or synthetic polyomers, including cellulose acetate, polysulfone, polycarbonate, polyamide, and polyacryl-polyamide acrylate.

An iontophoresis system locally delivers drug through a target site of internal body tissue, The iontophoresis system comprises a source electrode and a counter electrode in electrical communication with the source electrode. The counter electrode is configured to cooperate with the source electrode to form a localized electric field at the target site. A fluid cargo includes the drug and is capable of being delivered through the tissue of the target site when exposed to the localized electric field formed between the source electrode and the counter electrode. A surgically implantable reservoir is adapted to be secured to the target site. The reservoir comprises a housing having an inner surface defining an enclosed chamber and an inlet opening and an outlet opening for cargo flow through the chamber. The housing is capable of interacting with the localized electric field to release the cargo. A fluid channel extends from the outlet opening to the exterior of the housing. A platform extends inwardly into the chamber from the inner surface of the housing such that the platform and an adjacent portion of the inner surface of the housing define a trough surrounding the platform. The platform is configured for holding the source electrode. Means are provided for securing the housing to the tissue of the target site. Cargo flows through the reservoir from the inlet opening to the outlet opening moves gas bubbles formed by electrolysis from the surface of the electrode and carries the bubbles through the outlet opening and not the channel exterior of the housing.

The source electrode may comprise platinum. The cargo may comprise anesthetics, vaccines, chemotherapeutic agents, metabotites, immunomodutators, antioxidants, antibiotics, and ion channel regulators, or hormones. The cargo may also comprise one or more pharmaceutically acceptable carriers, excipients, or diluents or a therapeutic agent such as gemcitabine. The outlet opening may be spaced from the inlet opening including opposite the inlet opening. The housing securing means comprises a skirt around at least a portion of the reservoir, wherein the skirt may be sutured to tissue at the target site. The skirt may comprise a plurality of anchor points defining suture openings. The housing securing means can also comprise a biological adhesive. At least a portion of the housing comprises a membrane, the membrane allowing drug to pass through the membrane and into the targeted tissue when a localized electric field is applied. The membrane may comprise natural or synthetic polyomers, including cellulose acetate, polysulfone, polycarbonate, polyamide, or polyacryl-polyamide acrylate.

A surgically implantable reservoir is provided for implantation into a patient for use in an iontophoresis system for local delivery of ribonucleic acid molecules through a target site of internal body tissue. The iontophoresis system includes a source electrode and a counter electrode in electrical communication with the source electrode for forming a localized electric field at the target site. The reservoir comprises a housing having an inner surface defining an interior cavity open on one side for accommodating the electrode. A membrane spans the open side of the housing for covering the cavity in the housing forming an enclosed chamber. The membrane is capable of interacting with the localized electric field for allowing RNA molecules to pass through the membrane and into the targeted tissue when the localized electric field is applied. An absorbent pad for holding the ribonucleic acid molecules is disposed within the chamber along with a buffer gel at least partially filling the chamber. A spring assembly biases the electrode into the buffer gel and toward the pad and the membrane. Means are provided for securing the housing to the tissue of the target site. The housing securing means may comprise a skirt around at least a portion of the reservoir, wherein the skirt may be sutured to tissue at the target site. The skirt can comprise a plurality of anchor points defining suture openings. Housing securing means may comprise a biological adhesive. The membrane may comprise natural or synthetic polymers, including cellulose acetate, polysulfone, polycarbonate, polyamide, or polyacryl-polyamide acrylate.

### BRIEF DESCRIPTION OF THE DRAWINGS

For a more complete understanding of the reservoir for use with an interventional drug delivery device and system, reference should now be had to the embodiments shown in the accompanying drawings and described below.

In the drawings:

FIG. 1 is a schematic perspective view of an embodiment of an implantable reservoir for drug delivery attached to an anterior surface of a human pancreas.

FIG. 2A is an exploded left side perspective view of an embodiment of an implantable reservoir assembly for use with a system for interventional drug delivery to a target tissue site.

FIG. 2B is a transverse cross-section of a tri-lumen tube for use in delivery of fluid to the reservoir assembly as shown in FIG. 2A.

FIG. 2C is an exploded right front perspective view of the reservoir assembly as shown in FIG. 2A.

FIG. 3 is a left side elevation view of the reservoir assembly as shown in FIG. 2A.

FIGS. 4A-4C are left side and right side longitudinal cross-section views, respectively, of the reservoir assembly as shown in FIG. 2A.

FIG. 5 is a bottom plan view of the reservoir assembly as shown in FIG. 3 with the membrane removed.

FIG. 6A is a top perspective view of an embodiment of a reservoir for use with the reservoir assembly as shown in FIG. 2A.

FIG. 6B is a partially exploded top perspective view of an embodiment of a reservoir for use with the reservoir assembly as shown in FIG. 6A.

FIG. 6C is a partially cut-away top perspective view of an embodiment of a reservoir for use with the reservoir assembly as shown in FIG. 6B.

FIG. 7A is a bottom perspective view of the reservoir as shown in FIGS. 6A-6C including a membrane.

FIG. 7B is a bottom perspective view of the reservoir as shown in FIG. 7A with the membrane removed.

FIG. 8 is a bottom plan view of the reservoir as shown in FIGS. 6A-6C with the membrane removed and electrode in place.

FIG. 9 is a left side elevation view of the reservoir as shown in FIGS. 6A-6C, the right side elevation view being a mirror image thereof.

FIG. 10 is a left side longitudinal cross-section view of the reservoir as shown in FIG. 9.

FIG. 11 is a bottom plan view of the reservoir as shown in FIGS. 6A-6C with the membrane and electrode removed.

FIG. 12 is a top plan view of the reservoir as shown in FIGS. 6A-6C.

FIG. 13 is a front proximal end elevation view of the reservoir as shown in FIGS. 6A-6C.

FIG. 14 is rear distal end elevation view of the reservoir as shown in FIGS. 6A-6C.

FIG. 15 is a top plan view of the reservoir assembly as shown in FIG. 2A.

FIG. 16 is a longitudinal cross-section view of the reservoir assembly taken along line 16-16 of FIG. 15.

FIG. 17 is a partially exploded perspective view of a portion of the distal end of the tri-lumen tube.

FIG. 18 is a transverse cross-section view of a tri-lumen tube connector for delivery and removal of fluid to and from the reservoir assembly as shown in FIG. 2A.

FIG. 19 is a longitudinal cross-section view of the connection of the proximal end of the tri-lumen tube and the distal end of an external electrofluidic connector.

FIG. 20 is an exploded perspective view of an embodiment of an external electrofluidic connector for use with an implantable reservoir for delivery of therapeutic agent to a target site as shown in FIG. 1.

FIG. 21 is an elevation view of a distal end of the EFC as shown in FIG. 15.

FIG. 22 is a schematic view of the assembled implantable reservoir assembly and electrofluidic connector.

FIG. 23 is an exploded perspective view of an embodiment of a tunneler for placing an implant for delivery of therapeutic agent to a target site as shown in FIG. 1

FIG. 24 is an exploded perspective view of an embodiment of a cap for the EFC.

FIG. 25 is a schematic side perspective view of another embodiment of an implantable reservoir for use with a system for interventional drug delivery to a target tissue site.

FIG. 26 is a side elevation view of the implantable reservoir as shown in FIG. 25.

FIG. 27 is an exploded perspective view of the implantable reservoir as shown in FIG. 25.

FIG. 28 is a schematic cross-section view showing a fluid flow path for the implantable reservoir shown in FIG. 25.

FIG. 29 is a schematic cross-section view of another embodiment of an implantable reservoir showing an internal fluid flow path.

FIG. 30 is a top perspective view of an embodiment of a system including a reservoir for interventional drug delivery to an external target tissue site.

FIG. 31 is an exploded perspective view of the drug delivery system as shown in FIG. 30.

FIG. 32 is a side elevation view of the drug delivery system as shown in FIG. 30.

FIG. 33A is a longitudinal cross-section view of the drug delivery system as shown in FIG. 30.

FIG. 33B is a close-up view of a portion of the drug delivery system as shown in FIG. 33A.

FIG. 34A is a side elevation view of a reservoir for use view of the drug delivery system as shown in FIG. 30.

FIG. 34B is a longitudinal cross-section view of the reservoir as shown in FIG. 34A taken along line 34B-34B.

FIG. 35A is a top plan view of the reservoir as shown in FIG. 34A.

FIG. 35B is a longitudinal cross-section view of the reservoir as shown in FIG. 35A taken along line 35B-35B.

FIG. 36 is a top perspective view of an embodiment of a system for iontophoretic delivery of therapeutic ribonucleic acids (RNA).

FIG. 37 is an exploded top perspective view of the RNA delivery system as shown in FIG. 36.

FIG. 38 is a partially exploded top perspective view of the RNA delivery system as shown in FIG. 37.

FIG. 39 is a side elevation view of the RNA delivery system as shown in FIG. 36.

FIG. 40 is a longitudinal cross-section view of the RNA delivery system as shown in FIG. 36 taken along line 40-40 of FIG. 39.

FIG. 41 is an up-close longitudinal cross-section view of a portion of RNA delivery system as shown in FIG. 40.

### DESCRIPTION

Certain terminology is used herein for convenience only and is not to be taken as a limiting. For example, words such as “upper,” “lower,” “left,” “right,” “horizontal,” “vertical,” “upward,” “downward,” “top” and “bottom” merely describe the configurations shown in the FIGs. Indeed, the components may be oriented in any direction and the terminology, therefore, should be understood as encompassing such variations unless specified otherwise. The words “interior” and “exterior” refer to directions toward and away from, respectively, the geometric center of the core and designated parts thereof. The terminology includes the words specifically mentioned above, derivatives thereof and words of similar import.

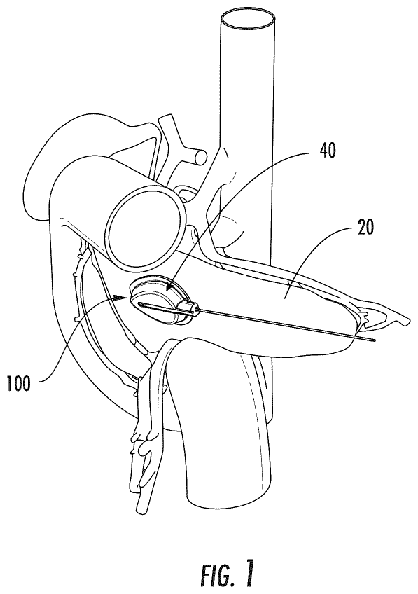

Referring now to the drawings, wherein like reference numerals designate corresponding or similar elements throughout the several views, an embodiment of a device and system for drug delivery at an internal site for therapeutic treatment using iontophoresis is shown in FIG. 1 as disposed on a surface of a human pancreas 20.

The iontophoresis system includes an embodiment of an implantable device for targeted drug delivery, which is generally designated at 100. The implant 100 comprises a reservoir assembly 40, including a reservoir body 42 for containing a therapeutic drug for delivery for treatment.

The reservoir body 42 is configured to receive a platinum electrode 90 for contacting a drug solution within the reservoir body. The reservoir body 42 is connected to a tri-lumen tube 44 having a proximal end 46 externally of the body of the patient and a distal end 48 connected to the reservoir body 42 and extending from the reservoir to the proximal end 46. Passing through the length of the tri-lumen tube 44 are an inlet conduit 50 for fluid flow into the reservoir 42, an outlet conduit 52 for fluid flow out of the reservoir, and a channel 54 for passing an electrical wire 56 for connection to the electrode 90.

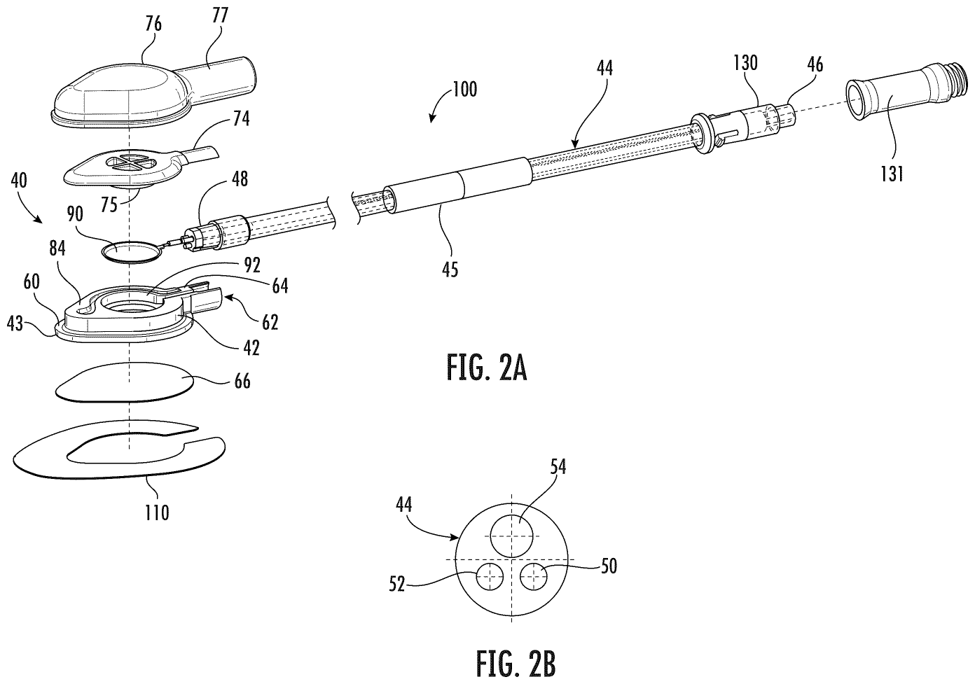

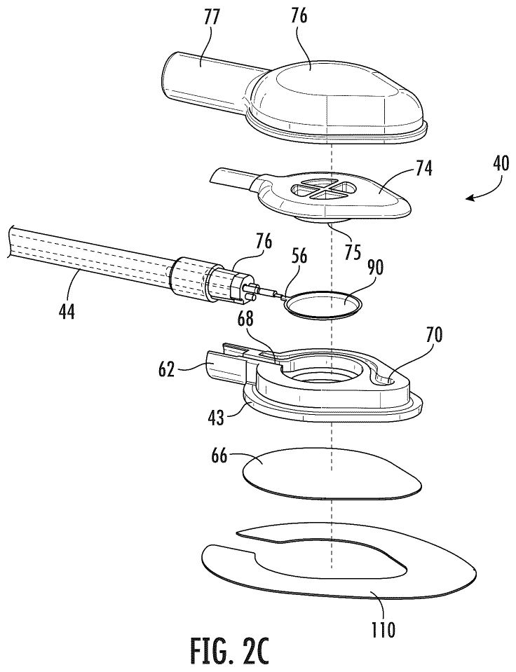

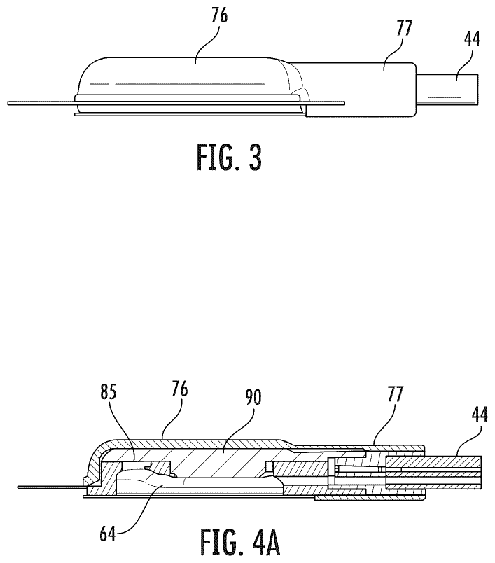

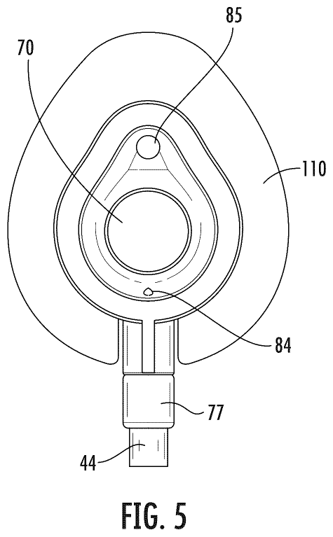

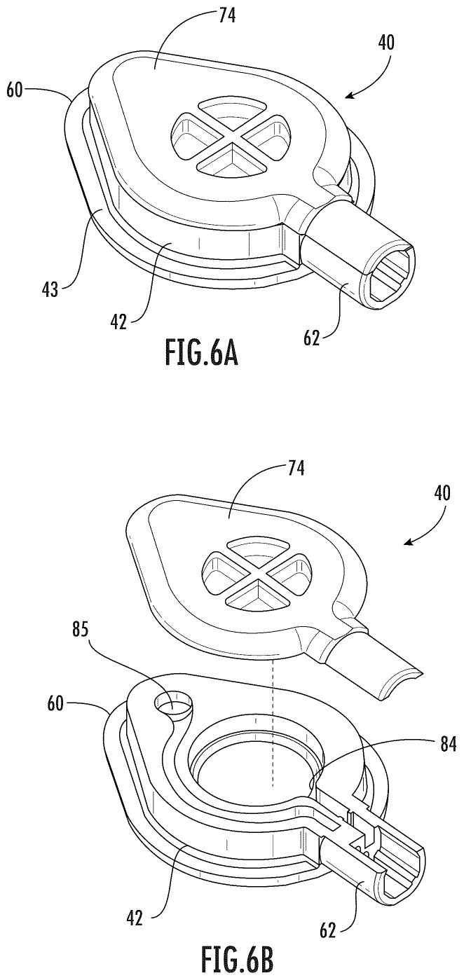

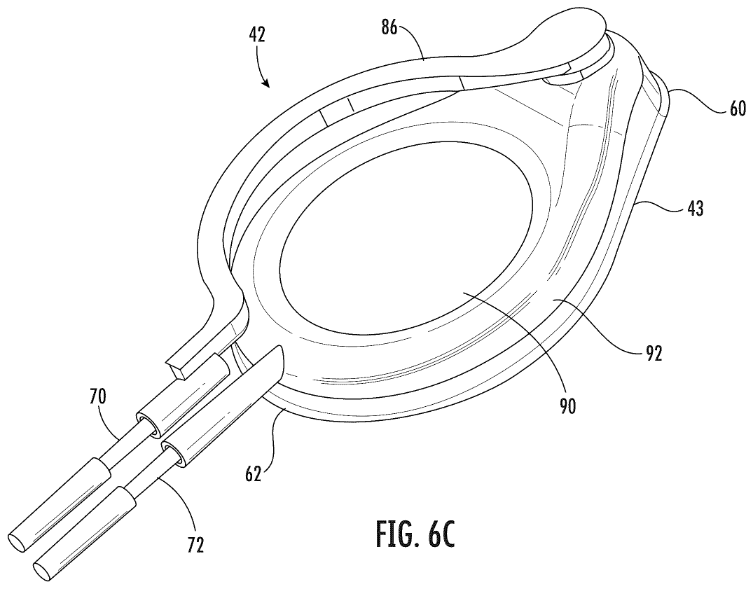

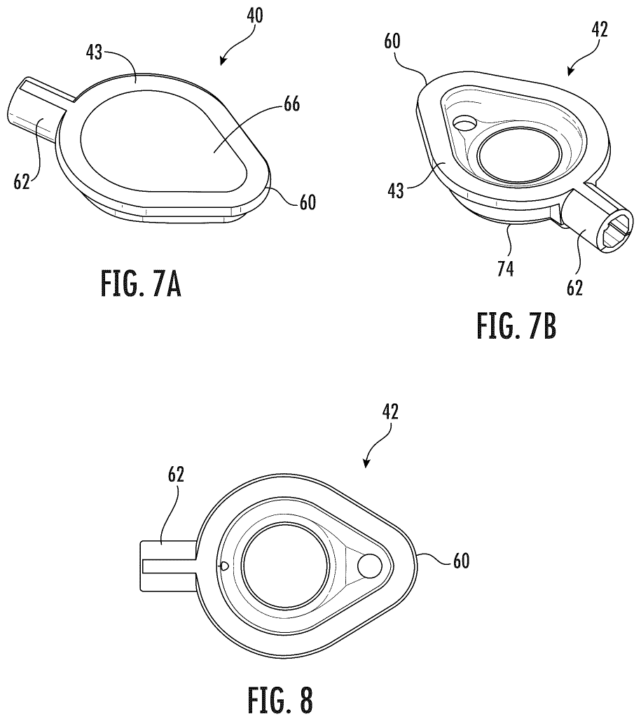

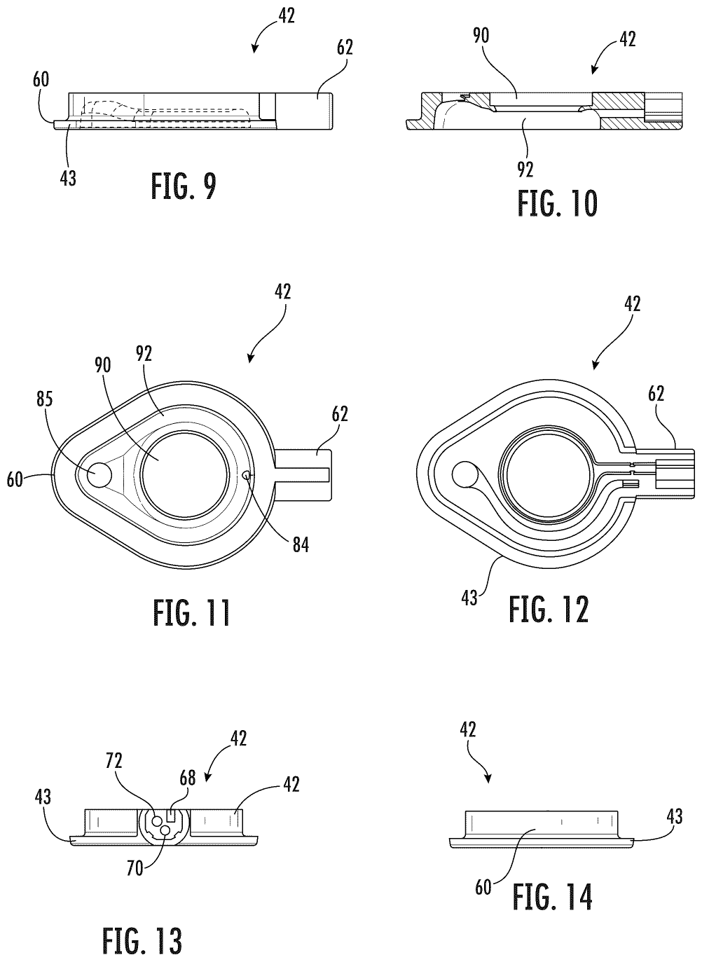

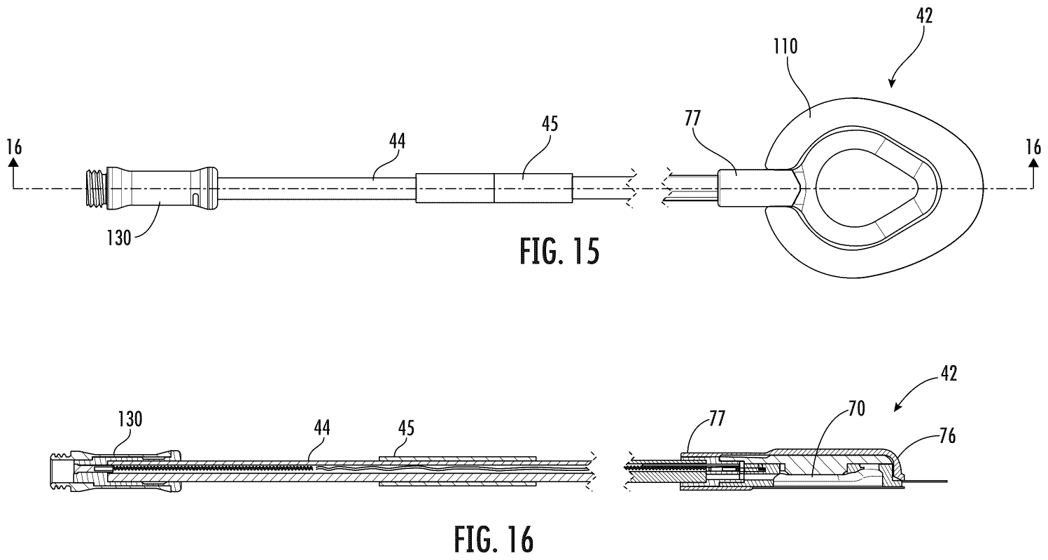

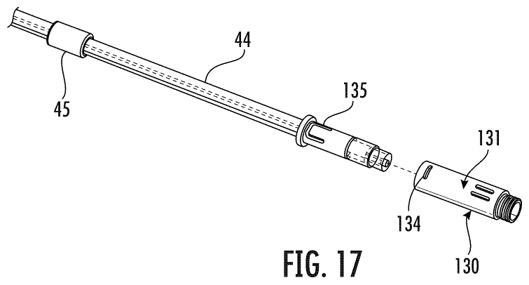

The reservoir assembly 40 is shown in FIGS. 2A-14. The reservoir body 42 is generally shaped in the form of an arrowhead, including a distal end portion 60 and a tubular proximal protrusion 62. The reservoir body 42 defines a centrally disposed circular cavity 64 opening downwardly through a bottom side of the reservoir body 42. A semi-permeable membrane 66 spans the bottom of the reservoir body 42. The membrane 66 is laser welded to the bottom surface of a rim 43 on the reservoir for covering and sealing the cavity 64 opening partially transforming the cavity 64 into a chamber defined by the reservoir body 42 and the membrane 66.

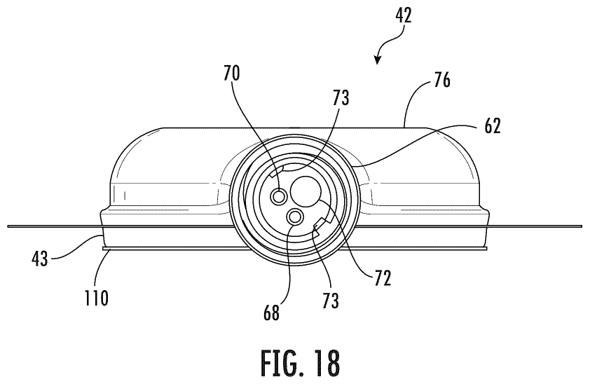

The proximal end protrusion 62 of the reservoir body 42 extends axially from the reservoir body 42. The proximal protrusion 62 defines three axial passages, two of which open into the chamber 64 in the reservoir body 42. Referring to FIG. 18, the three axial passages defined by the proximal end protrusion 62 include an inlet passage 68 and an outlet passage 70 for fluid, and a channel 72 for passing the electric wire 56 to the electrode 90. A pair of oppositely disposed tabs 73 outwardly radially spaced from the axial passages 68, 70, 72 function as an alignment and locking means for receiving the distal end 48 of the tri-lumen tube 44.

The reservoir body 42 has a top portion 74 having a shape corresponding to the shape of the reservoir body 42. The top portion 74 includes an inwardly depending circular plug 75. The top portion 74 is laser welded onto the reservoir body 42 such that the plug 75 secures the electrode 90 into position without the need for adhesive on the electrode. This arrangement is fluid tight so that electrolysis can only occur inside the reservoir body 42.

A cap 76 correspondingly shaped to the reservoir body 42 fits over the top side of the reservoir body 42 providing a protective covering. The cap 76 includes a corresponding proximal protrusion 77, forming a sealed chamber. The proximal protrusion 77 is configured to cover the proximal protrusion 62 of the reservoir body 42. The outer surface of the cap 76 is smooth so that it does not affect adjacent organs or other body tissue. The upper surface of the proximal protrusion 62 defines a first linear groove 80. The groove 80 extends along the length of the protrusion 62 and terminates distally at an opening 84 into the chamber 64 in the reservoir body 42. The linear groove 84 is in fluid communication with the inlet passage 68 in the proximal end protrusion 62. The upper surface of the proximal protrusion 62 and the contiguous upper surface of the body portion of the reservoir body 22 define a second curved groove 82 spaced outwardly from the opening in the reservoir body 42. The curved groove 82 terminates distally in an outlet opening 85 through the reservoir body 42 and into the chamber 64 defined by the reservoir body 42, the membrane 66 and the top portion 74. The curved groove 82 is in fluid communication with the outlet passage 70 in the proximal end protrusion 62. This arrangement provides fluid communication between the outlet conduit 52 in the tri-lumen tube 44 and the chamber 64. The inlet opening 84 in the reservoir body 42 is directly opposite the outlet opening 85 along a longitudinal axis including the proximal protrusion 62.

The proximal protrusion 62 is configured for a sealed connection to the distal end 48 of the tri-lumen tube 44 such that the passages 50, 52, 54 in the tri-lumen tube 44 are in fluid communication with the passages 68, 70, 72 in the reservoir body 42 through to the chamber 64. The implant connector comprises a clocking feature to ensure alignment between all of the corresponding passages thereby allowing the user to connect correctly every time.

The reservoir body 42 and the cap 76 may be formed from polysulfone. It is understood that the reservoir may be formed from any other soft flexible material that is also biocompatible. The membrane 66 may comprise natural or synthetic polymers including, but not limited to, polycarbonate, polyethylene terephthalate (PET). polyamide, or polyacryl-polyamide acrylate. Organic membranes can include polyethersulfone (PES), polyacrylonitrile (PAN), polytetrafluoroethylene (PTFE), sulfonated tetrafluoroethylene copolymer (Nafion), polyamide-imide (PAI), and polyvinylidenedifluoride (PVDF), polyphenylene oxide (PPO), polystyrene, nylon, polyether ether ketone (PEEK), hydrophilic and hydrophobic polyester (PETE), or polypropylene. Natural polymers may include natural rubber and cellulose (cellulose acetate).

The platinum electrode 90 is placed on a platform 92 integral with the center of the body of the reservoir 42. The electrode 90 is circular and generally planar. The outer edge of the electrode 90 is angled upwardly and crimped so that the electrode resembles a “pie pan”. This shape eliminates electrolysis nucleation sites, which decreases bubble formation. The electrode 90 may be coated with tin, titanium nitride, to further reduce the possibility of bubble formation during iontophoresis. The platform 92 extends inwardly from an inner surface of the body of the reservoir 42 and into the chamber 64. The walls of the platform 92 are spaced inwardly from the adjacent inner surface of the reservoir body 42 forming a trough 94 surrounding the platform 92 and electrode 90. The electrode 90 is connected to a power source via the platinum wire 56 extending the length of the tri-lumen tube 44 and the reservoir body 42. A gold plated pogo pin 126 connector and pad 127 allow connection at points allow the 56, as will be described below.

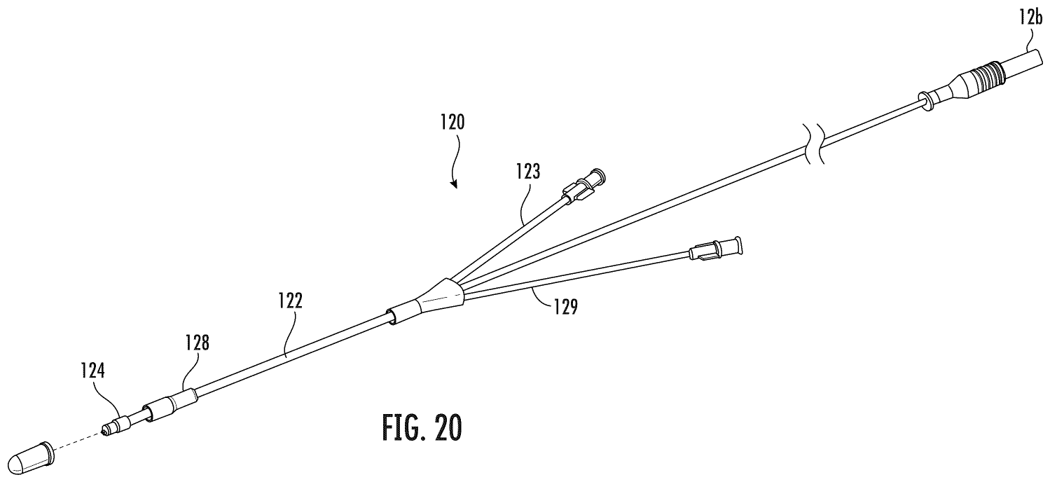

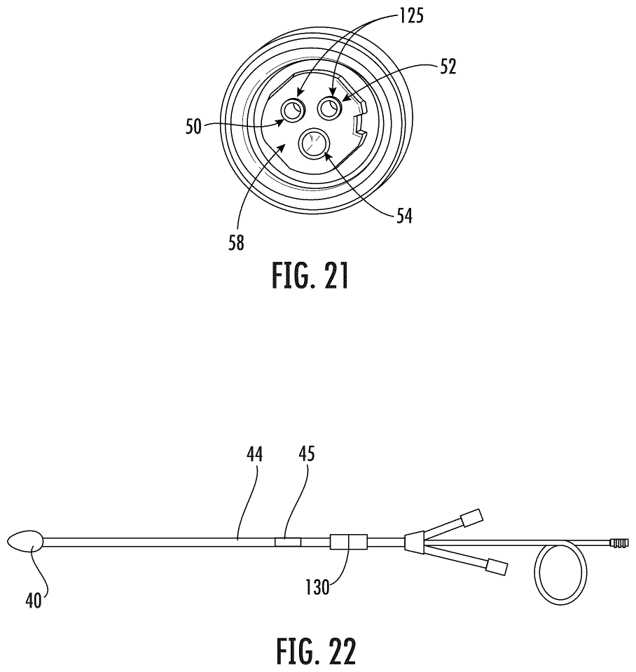

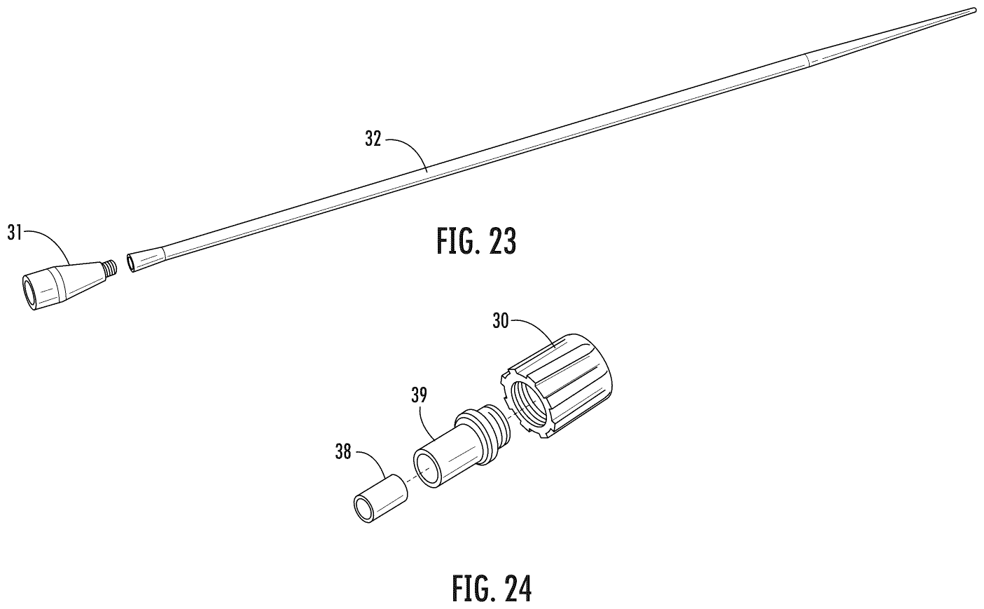

The tri-lumen tube 44 is connected at the proximal end 46 externally of the body of the patient to an electro fluidic connector (EFC) 120, shown schematically in FIG. 20 by means of a hybrid connector 130. The tri-lumen tube 44 has a protective dacron cuff 45 (FIG. 2A) over a section where the tri-lumen tube 44 is meant to be disposed inside a subcutaneous tunnel on the flank of a patient. Dacron provides for tissue ingrowth and prevents microbes or viruses from migrating down the tube and into the patient. Dacron is radio opaque and easily identifiable under X-Ray or CT imaging.

The EFC 120 is a part of an external disposable subsystem that is connected via a distal end connector 124 to the implantable device for therapeutic drug delivery for treatment via the hybrid connector 130. The EFC 120 includes a tri-lumen tubular conduit 122 comprising the connector at the distal end 124. This connector 124 mates with the implantable device connector 130 sealing the fluid and electrical channels within the tri-lumen tube 44 for passing fluid and the electrical wire. An intravenous IV infusion pump set (not shown) and a waste bag (not shown) are connected to the inlet conduit 123 and the outlet conduit 129, respectively. The fluid enters the inlet conduit 123 of the EFC 120 and connects through its distal end connector 124 to the implantable device's connector 130, communicating with the inlet conduit 50 of the tri-lumen tube 44 via the connection. The IV infusion pump moves fluid through inlet tube 123 of the EFC, through the inlet conduit 50 and through the implant reservoir 42 and out through the outlet conduit 52 of the tri-lumen tube 122, and out through the outlet conduit 129 of the EFC 120 and into the waste bag when flushing and priming the implanted drug delivery device 100. The electrical wire 56 terminates in an electrical connector at a proximal end of the EFC 120 for accessing an external power supply.

During treatment of the pancreas or other internal site, a drug solution is pumped through the EFC 120 and the drug delivery device 100. Alternatively, the inlet conduit 123 and the outlet conduit 129 of the EFC 120 may be connected to a drug solution bag (not shown). The drug solution is circulated out of the bag into the EFC 120, through the drug delivery device, and back into the drug solution bag. A return, or counter, electrode (not shown) is placed on the subject's back directly behind the pancreas or otherwise adjacent to the internal delivery site. There may be a plurality of counter electrodes as needed. For example, there may be up to up to five or more counter electrodes at selected locations on the body of the subject. The counter electrodes are electrically connected to direct a current (DC) power supply and a controller for completing the circuit. The power supply provides a specified voltage limited, constant current during treatment.

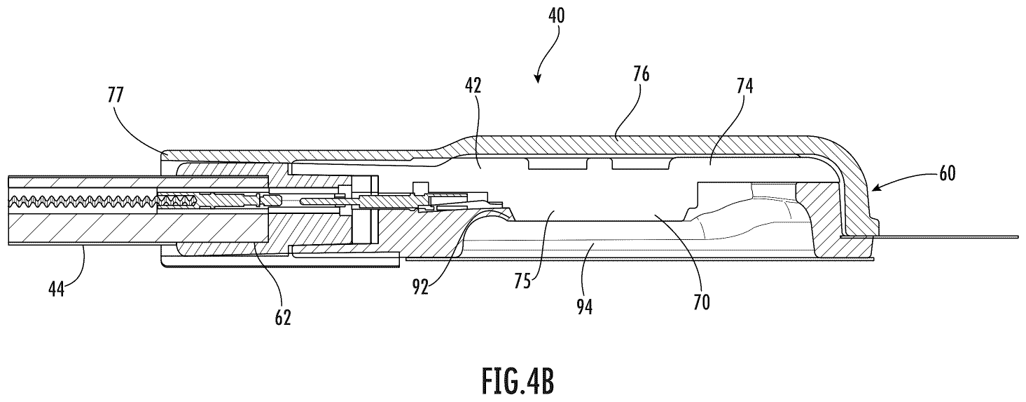

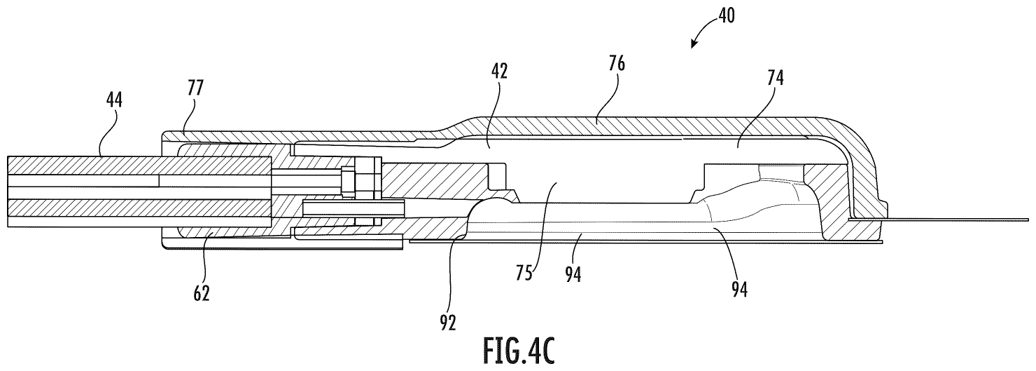

The distal end 48 of the tri-lumen tube 44 includes an electrical connector and pin which fits through the tubular proximal protrusion from the cap 76 and butts against the end of the proximal protrusion 62 from the reservoir body 42 (FIG. 4). The distal ends of the inlet and outlet conduits 50, 52 extend distally from the tri-lumen tube 44 into the passage and the grooves 80, 82 respectively, and are sealed against the reservoir through an elastic flange. A fixation skirt 110 is captured between the reservoir body 42 and the cap 76. The fixation skirt 110 is formed of bio-compatible polyester mesh which provides openings for suturing the reservoir body 42 to body tissue for securing the reservoir 42 to the target site in the body. Alternatively, the reservoir body 42 may be fixed to a target site of body tissue using a biological adhesives, microneedles utilizing a ring at the bottom of the reservoir body, or staples, either alone or in combination with sutures.

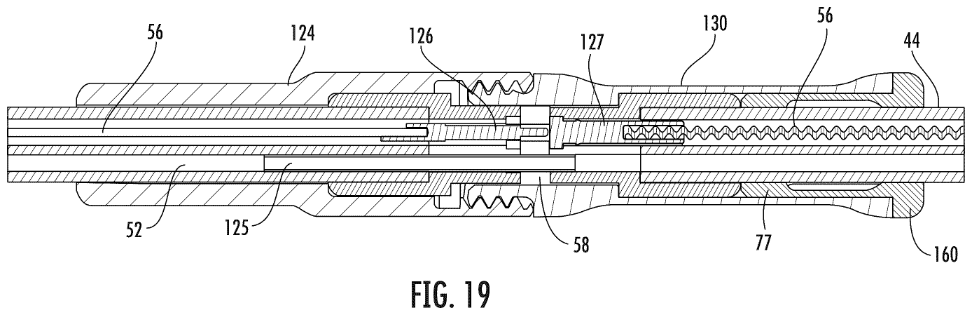

The proximal end 46 of the tri-lumen tube 44 comprises a hybrid connector 130 and a slidable cover 131 that is externalized after implantation of the reservoir body 42. The hybrid connector 130 is configured to mate with the distal end 124 of the EFC 120 for providing fluid and electrical communication. More specifically, the inlet conduit 50 and the outlet conduit 52 of the tri-lumen tube 44 extending from the reservoir body 42 terminate at their proximal ends with the EFC 120 in fluidic connectors on the inlet and outlet conduits 52, 54, respectively. A pogo pin connector 126 provides for electrical connection and to also ensure proper alignment of the conduits 52, 54 for fluid communication. Referring to FIG. 19, the connection between the EFC 120 and the implantable device 100 is shown through the mating connection of the distal end 124 of the EFC and the hybrid connector 130 at the proximal end 46 of the tri-lumen tube 44, which is in fluid communication with at the distal end 48 with the proximal end 62 of the reservoir 42. The distal end connector 124 of the EFC 120 comprises two stainless steel hypotubes 125, each protruding from within the inlet conduit 50 and the outlet conduit 52, respectively. The spring-loaded pogo pin 126 extends from the electrical channel 54. All three components 125, 126 pass through a flexible gasket, or gland, 58 of a soft material disposed at the distal end 124 of the EFC 120 to provide a seal when connected to the hybrid connector 130. The connector 130 comprises a retainer clip 160 for the metal connector 130 to prevent it from moving along the trilumen tube 44. The connector 130 terminates in a threaded end that is received in the internally threaded outer cover 128 of the distal EFC connector 124. The hybrid connector 130 has inlet and outlet fluid channels that receive the hypotubes 125. The electrical channel 54 containing the electrical wire 56 in the hybrid connector 130 terminates on a pogo pad 127 that contacts the pogo pin 126 from the EFC when the two connectors 120, 130 are threaded together. When the two connectors 120, 130 are screwed together, the gland 58 compresses and seals around the hypotubes 125, which are inserted into the fluidic channels of the protrusion 62. This arrangement provides fluid and electrical communication from external to the body of the patient to the reservoir assembly 42.

The proximal end of the inlet conduit 123 of the EFC may receive a luer wing fitting for controlling the flow of fluid into the EFC to the reservoir 42 via the inlet conduit. The proximal end of the outlet conduit 129 may also have a luer fitting. The fittings allow fluid to pass from the reservoir and out of the proximal end of the outlet conduit.

In use, the reservoir assembly 100 is implanted at a target site in the body of a patient. In the embodiment shown in FIG. 1, the reservoir assembly 100, including the reservoir body 42 containing drug solution and a source electrode 90, is secured to the anterior surface of a pancreas 20. The tri-lumen tube 44, including the inlet and outlet conduits 50, 52 and the electrical cable 56, emerges through the abdomen for connection to the EFC 120 and then to an infusion pump and power source, respectively. To complete the iontophoresis device and system, a second counter electrode (not shown) is placed on the skin of the patient, typically on the back when treating the pancreas, for completing the electrical circuit. A fluid cargo including the drug solution to be delivered is supplied through the inlet conduit 50 into the chamber 64 of the reservoir 42 via the EFC 120 and tri-lumen tube 44. An electrical field is generated between the source electrode 90 and the counter electrode for moving the drug across the membrane 66 and into the tissue of the pancreas and a tumor.

Electrolysis at the source electrode 90 causes gas bubbles to form, which adhere to the electrode surface. Flow of the cargo fluid through the reservoir body 42 from the fluid inlet opening 84 to the outlet opening 85 over the electrode 90 and around platform 92 in the trough 94 removes and carries the bubbles from the chamber 64 through the outlet opening 85. The trough 94 formed around the platform 92 holding the electrode 90 and the aligned fluid inlet opening 84 and outlet opening 85 create a flow pattern that effectively sweeps the bubbles off of the electrode surface and out of the chamber 64 of the reservoir body 42. The area of highest fluid flow rate is across the raised platform 92 on which the electrode 90 is positioned so that bubbles that form on the electrode are swept into the trough 94. The bubbles formed in the reservoir chamber 64 are thus carried off the electrode 90 by the fluid flow and carried into the trough. The bubbles accumulate at the outlet opening 85 in the distal end of the chamber 64 opposite to the inlet opening 84 prior to exiting. As seen in FIG. 4B, for example, the distal area of the chamber 64 adjacent the outlet opening 85 is elevated, creating a small dome where bubbles collect prior to evacuation. The bubbles are then carried through the opening 85 and out of the chamber 64 of the reservoir body 42 by the fluid flow though the channel 86 created above the reservoir 42. The channel 86 connects fluid flow to the outlet fluid passage 70 for the bubble evacuation.

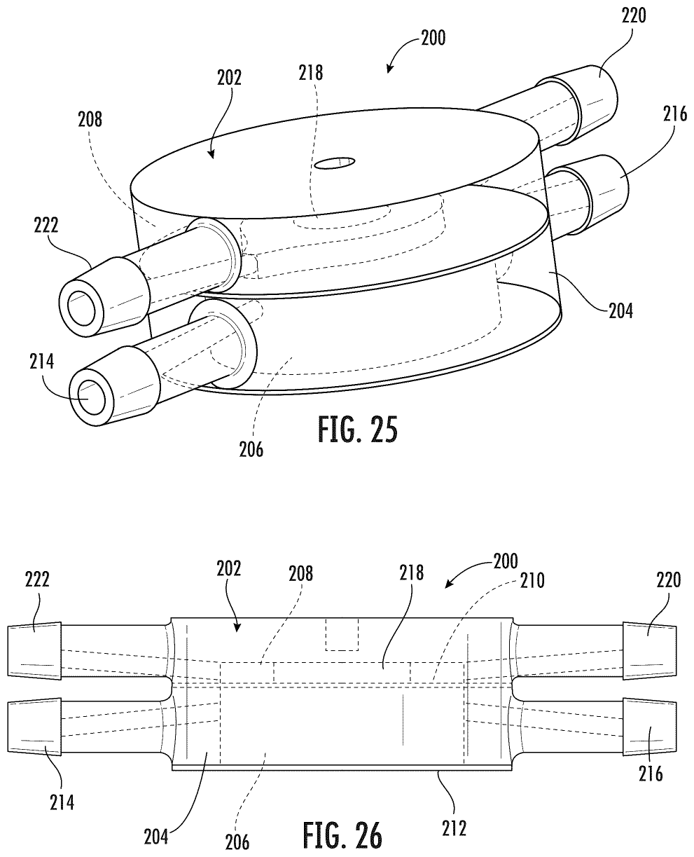

Referring now to FIGS. 25-27, another embodiment of an iontophoresis device and system for drug delivery at an internal target tissue site for therapeutic treatment is shown and generally designated at 200. This second iontophoresis device and system for drug delivery 200 includes an implantable dual reservoir assembly 202. The dual reservoir assembly 202 comprises a reservoir body 204 housing a lower chamber 206 and a separate upper chamber 208 for containing a therapeutic drug for delivery. The lower chamber 206 is defined by the reservoir body 204 and an upper semipermeable membrane 210 spaced from a lower semipermeable membrane 212. Each membrane 210, 212 spans the reservoir body 204 for sealing and forming the lower chamber 206 as defined by the reservoir body 204 and the membranes 210, 212. As such, the membranes 210, 212 provide a “floor” and a “ceiling”, respectively, of the lower chamber 206. An inlet orifice 214 and an outlet orifice 216 open into the lower chamber 206. The inlet orifice 214 is in fluid communication with a source of therapeutic drug solution.

The upper chamber 208 of the dual reservoir accommodates an electrode 218 for contacting a drug solution within the upper chamber 208. The electrode 218 also functions, at least partially, as a “ceiling” for the upper chamber 208. The “floor” for the upper chamber 208 is the semipermeable membrane 210 which also serves as the “ceiling” of the lower chamber 206 of the reservoir assembly 202. Thus, in this configuration the lower chamber 206 and the upper chamber 208 are physically separated by the membrane 210. The upper chamber 208 has an inlet orifice 220 in fluid communication with the outlet orifice 216 of the lower chamber 206 and an outlet orifice 222.

In the configuration shown in the drawings, the floor and ceiling of each of the lower chamber 206 and the upper chamber 208 are substantially parallel. The distance between the floor 210 and ceiling 218 of the upper chamber 208 is less than the distance between the floor 212 and ceiling 210 of the lower chamber 206. Therefore, in this arrangement, the upper chamber 208 has less volume than the lower chamber 206 resulting in a higher fluid flow rate through the upper chamber 208 than through the lower chamber 206. While the reservoir body 204 is shown to be generally cylindrical, it is understood that the reservoir is not so limited and the shape may be any suitable shape for the flow of fluid through the chambers 206, 208 and transfer and delivery of the drug solution.

In use, the reservoir assembly 202 is implanted at a target tissue site in the body of a patient. The lower membrane 212 is against the surface of the tissue. To complete the iontophoresis device and system, a second counter electrode (not shown) is placed on the skin of the patient for completing the electrical circuit. A fluid cargo including the drug solution to be delivered to target tissue, for example a chemotherapeutic drug solution or any other fluid polar substance, is pumped from external to the body through the inlet orifice 214 and into the lower chamber 206 of the reservoir 204. The fluid fills the lower chamber 206 and exits the outlet orifice 216 which is in fluid communication via external tubing (not shown) with the inlet orifice 220 of the upper chamber 208. Fluid fills the upper chamber 208 and exits the upper chamber 208 via the outlet orifice 222. Since the fluid flow rate is identical for the fluid passing through the lower chamber 206 and the lower volume upper chamber 208, the velocity of fluid flow is higher through the upper chamber 208.

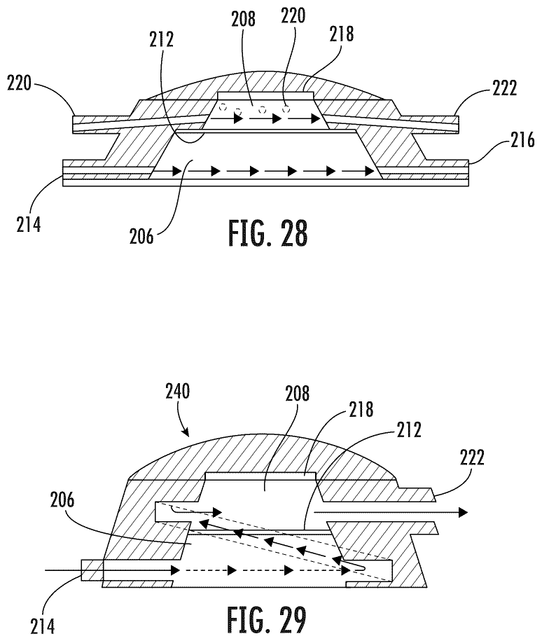

During iontophoresis (FIG. 28), an electrical field is generated between the source electrode 218 and the counter electrode for moving the drug across the membranes 210, 212 and into the target tissue. Electrolysis at the source electrode 218 in the upper chamber 208 causes gas bubbles to form. The gas bubbles adhere to the electrode 218 surface at nucleation sites, which generate gas as part of electrolysis. Bubbles on the surface of the electrode 218 reduce the active area of the electrode due to the difference in dielectric properties between liquid and gas. The presence of gas bubbles 220 thereby effectively reduces the area of the electrode 218 and create non-uniform distributions of the electric field. The higher fluid flow rate through the upper chamber 208 is designed to push gas bubbles generated during electrolysis from the vicinity of the electrode 218 and out of the upper chamber 208 through the outlet orifice 222. The bubbles formed in the upper chamber 208 are thus carried off the electrode 218 by the fluid flow and out of the upper chamber 208. Removing the bubbles functions to maintain a more uniform electric field thereby avoiding circumstances wherein a bubble 224 grows on the surface of the electrode 218. This condition can lead to the electric field which may not be evenly distributed with current density changes creating a higher intensity where the electrode 218 is free of bubbles compared to the side where the bubbles are present.

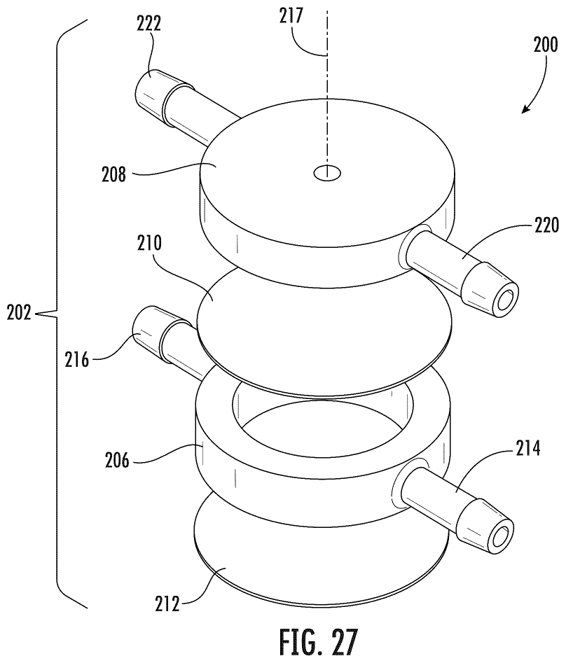

Another embodiment of an implantable dual reservoir assembly 202 is shown in FIG. 29 and generally designated at 240. In this embodiment, the lower chamber 206 is in fluid communication with the upper chamber 208 via an internal channel 242 that climbs with an upward trajectory along a semicircular path from the outlet orifice 216 of the lower chamber 206 to the inlet orifice 220 of the upper chamber 208. It is understood that the location and arrangement of the inlet and outlet orifices to the upper chamber and the lower chamber are flexible. For example, the inlet and the outlet may be placed vertically from one another. Alternatively, a single multichannel connector may be connected to each chamber. The connector can also have a third channel that carries an electrical conductor to energize the electrode. In the present embodiment, an electrical wire 219 to deliver power to the electrode 218 may pass through the body 204 of the reservoir to the electrode 218 (FIG. 27).

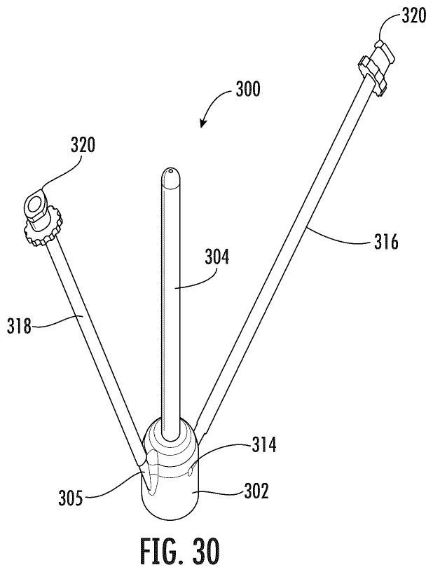

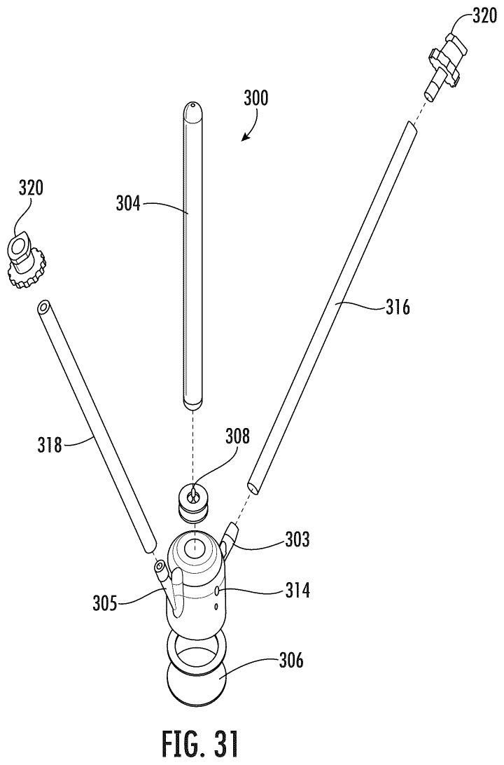

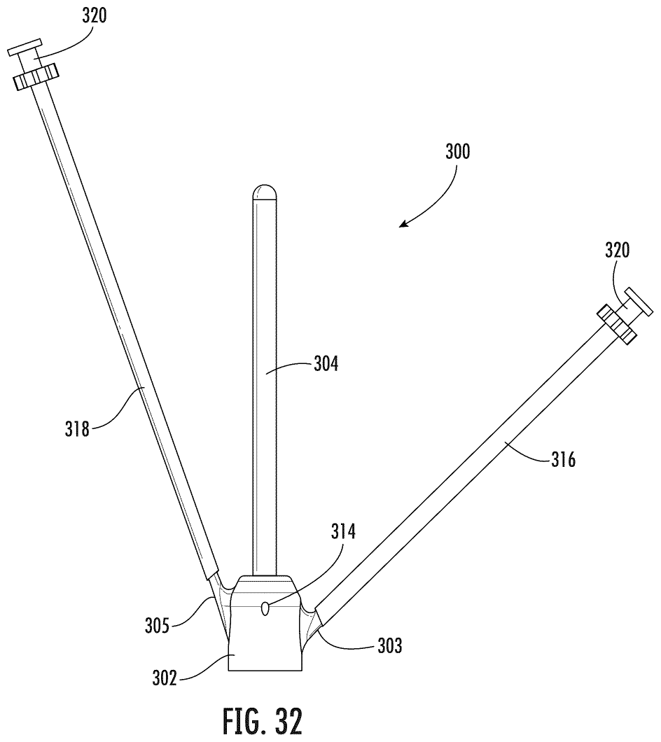

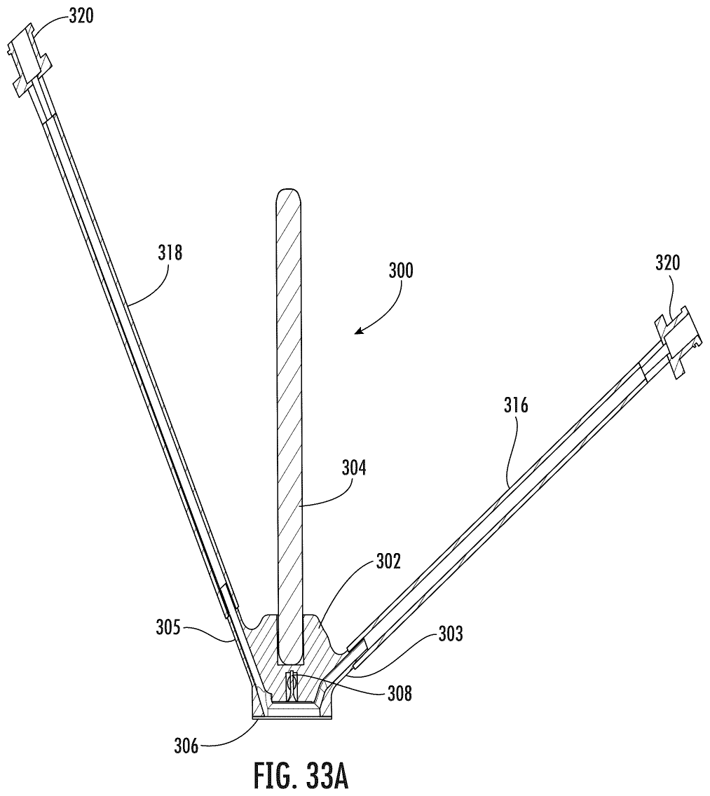

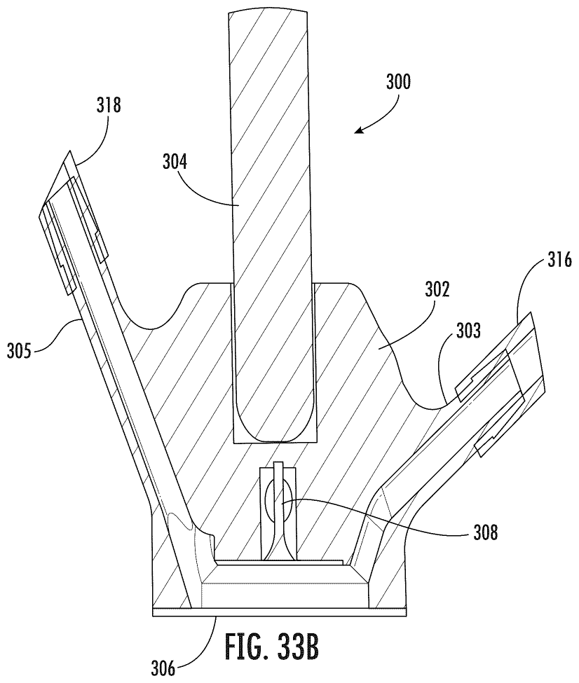

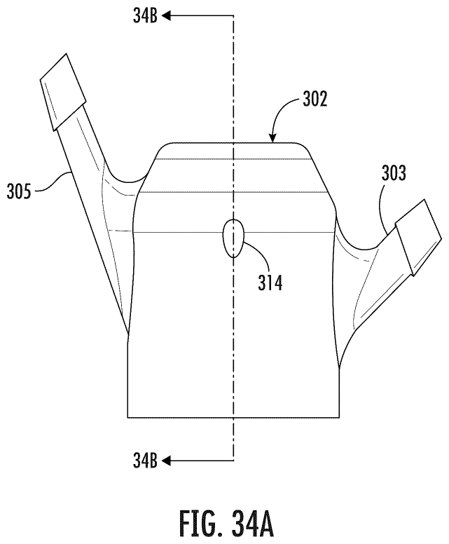

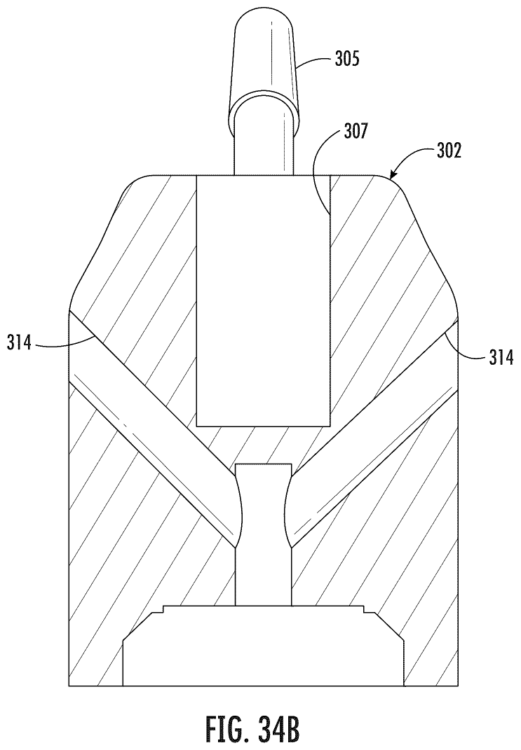

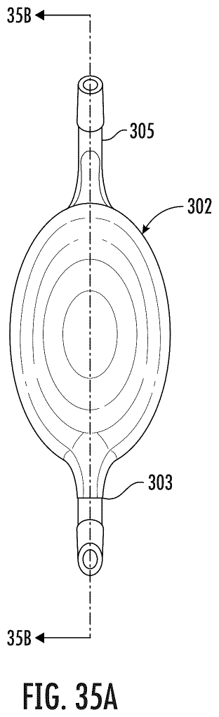

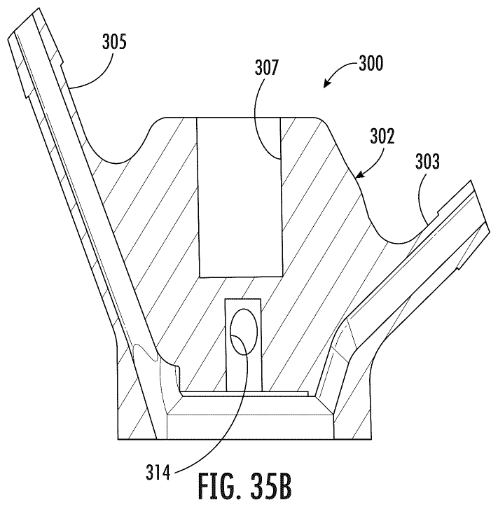

Referring now to FIGS. 30-33B, an embodiment of an iontophoresis device and system for targeted drug delivery at an external bodily tissue site for therapeutic treatment is shown and generally designated at 300. The external device 300 comprises a generally cylindrical reservoir base 302 having a fluid inlet 303 and a fluid outlet 305 and defining a cavity open to the bottom of the reservoir base 302. A semi-permeable membrane 306 spans the bottom of the reservoir base 302 for covering and sealing a chamber 307 defined by the reservoir base 302 and the membrane 306. The inlet 303 and the outlet 305 open into the chamber 307. An electrode 308 is bonded with epoxy or PSA adhesive to the ceiling of the reservoir base 402 opposite the membrane 306. A wire 309 is spot welded or soldered to the electrode 308 and the top of the reservoir base 302 and exits through a side orifice 314 (FIGS. 35A and 35B). The orifice 314 also serves as a filling hole to pot with epoxy and prevent fluid from migrating to the back of the electrode 308 (FIGS. 34A and 34B). One embodiment of the external device 300 has a handle 304 to hold the device in a fixed position in space during treatment while pressed against target tissue in an area of the body. Areas of the body that may be treated include, but are not limited to, the oral cavity, face, neck, and the like.

In use, the reservoir base 302 is held by the handle 304 at a target tissue site on the body of a patient with the membrane 306 against the surface of the tissue. To complete the iontophoresis device and system, a second counter electrode (not shown) is placed on the skin of the patient for completing the electrical circuit. A fluid cargo including the drug solution to be delivered to the target tissue, for example a chemotherapeutic drug solution or any other fluid polar substance, flows from a source and via tubing 361 and through the inlet 316 into the lower chamber 307 of the reservoir base 302. The fluid fills the chamber 307 and exits via the outlet 305 which is in fluid communication to external tubing 318.

During iontophoresis, an electrical field is generated between the source electrode 308 and the counter electrode for moving the drug across the membrane 306 and into the target tissue. As described above, electrolysis at the source electrode 308 causes gas bubbles to form, which adhere to the electrode surface. Fluid enters via the fluid inlet 303 and passes over the electrode 308 to the outlet 305. The outlet 305 is steeply angled relative to the reservoir base 302 to improve the evacuation of the bubbles that may form from electrolysis. For example, the outlet may extend upwardly and outwardly form the reservoir base 302 at an angle of between about 30 degrees to about 90 degrees relative to a horizontal plane. Ideally, this angle will allow the outlet 305 and associated tubing 316 to be positioned at or near vertical during use. The fluid pushes the bubbles across the electrode 308 towards the outlet 305. The near vertical outlet 305 acts as a chimney for the bubbles while drug is simultaneously being delivered to the target tissue.

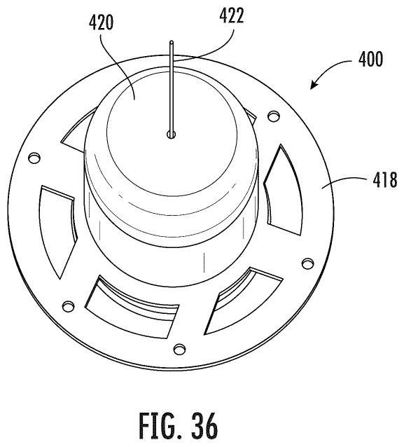

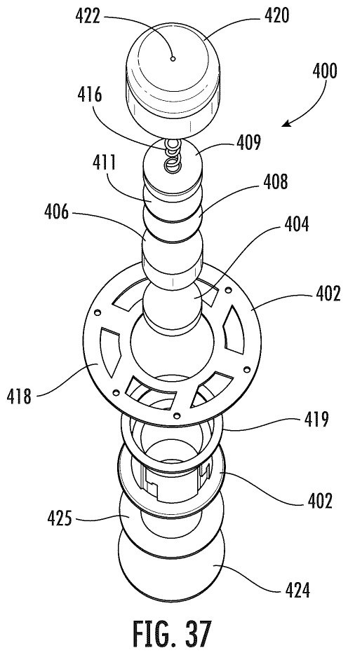

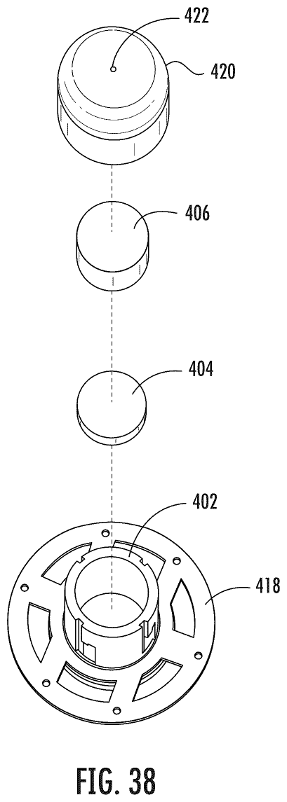

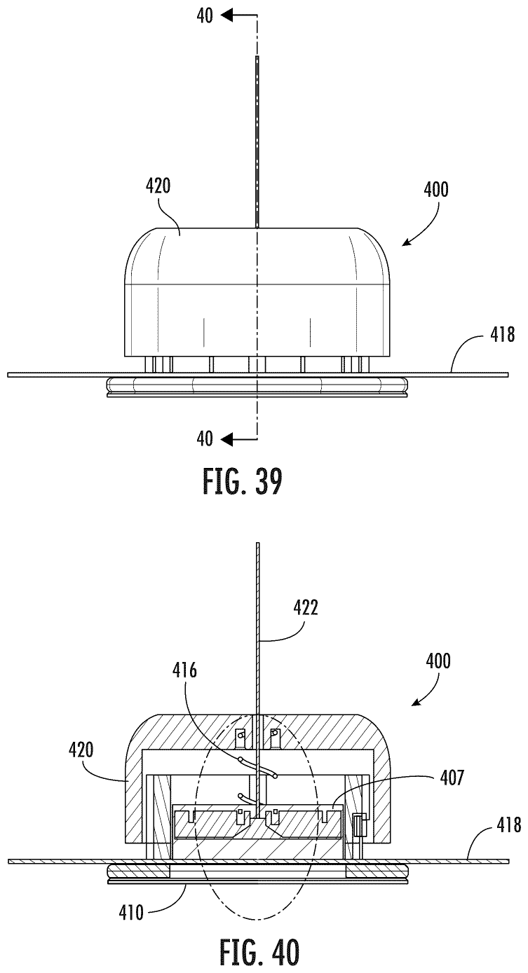

An embodiment of an implantable device and system for iontophoretic delivery of therapeutic ribonucleic acids is shown in FIGS. 36-41 and generally designated at 400. The ribonucleic acids (RNA's) delivered include, but are not limited to, siRNA, miRNA, shRNA, mRNA, and related species. The RNA delivery system 400 is designed to stabilize RNA molecules during iontophoretic transport and to provide consistent, reproducible delivery into target tissue site in the body. The RNA delivery system 400 comprises a slotted reservoir 402 that houses an RNA pad 404, a buffer gel 406, and a platinum electrode 408. The slotted reservoir 402 functions as the container for the other listed functional components and provides alignment and spacing of the components for proper ionic conduction. In particular, the configuration of the slotted reservoir 402 provides secure positioning of the electrode 408 for current application.

The RNA pad 404 is configured to load, absorb, and retain aqueous formulations of the ribonucleic acids prior to iontophoretic delivery. The RNA pad 404 may be formed from woven or non-woven textile substrates, sponges, or other porous matrices capable of loading, absorbing, and retaining the aqueous solutions of RNA molecules. The RNA pad 404 may hold a defined dose of RNA solution. During operation, the RNA 404 pad serves as a reservoir positioned within the iontophoretic transport pathway. In this arrangement, the RNA pad 404 facilitates controlled release and availability of RNA molecules for migration into the target tissue under the applied electric field.

The buffer gel 406 is positioned within the reservoir in contact with the RNA pad 404. The buffer gel 406 is essential for maintaining stability of the nucleic acid cargo and functions to neutralize pH fluctuations resulting from electrolytic processes at the electrode 408. During iontophoresis, electrolysis at the anode and cathode can generate hydrogen and chlorine gases and alter the local pH. Such fluctuations in pH can result in degradation, denaturation, or functional inactivation of the RNA molecules being delivered. The buffer gel 406 stabilizes these changes, protecting RNA integrity while maintaining ionic conduction. When used in combination with the RNA pad 404, the buffer gel 406 ensures that the RNA remains accessible for transport, while simultaneously protected from local pH fluctuations and electrolytic byproducts that could otherwise degrade or inactivate the RNA. The cooperative action of the RNA pad 404 and the buffer gel 406 thereby supports efficient ion conduction, stabilizes the therapeutic RNA, and promotes reliable delivery to the target tissue.

The electrode 408 interfaces with the buffer gel 406 and the RNA pad 404 to generate ionic transport when a voltage is applied across the system. The preferred electrode 408 is platinum in the form of a thin disk, approximately 1-25 mm in diameter and of sub-millimeter thickness. The electrode 408 drives electrical current during iontophoretic operation. Platinum is selected for its electrochemical stability, conductivity, and biocompatibility, allowing for reliable current delivery while minimizing degradation under electrolytic conditions. An electrode disk 409 may be included to support and position the platinum electrode 408 within the device assembly. The disk 409 ensures precise placement of the electrode 408 relative to the buffer gel 406 and RNA pad 404 allowing for uniform current distribution during iontophoretic transport. Adhesive may be used to secure the platinum electrode 408 to the electrode disk 409. The adhesive provides durable bonding under operational stresses, ensuring that the electrode 408 remains properly oriented and in conductive contact with the other device components throughout the delivery procedure.

A semipermeable membrane 410 is disposed on the tissue-facing side of the reservoir 402. The membrane 410 directly interfaces with the target tissue and functions as the final transport medium for RNA molecules delivered from the iontophoretic system 400, permitting selective directional passage of therapeutic RNA molecules while separating the internal components from the target tissue treatment site. The membrane 410 thus acts as the functional interface for therapeutic RNA delivery. An adhesive bonds the semipermeable membrane 410 to the reservoir 402 providing durable and fluid-tight integration of the membrane with the reservoir and surrounding structures, preventing leakage and maintaining controlled directional release of ribonucleic acid molecules through the membrane 410 and into the target tissue.

A ridged cap 420 seals the top of the reservoir 402 and is used to enclose the internal components within the slotted reservoir 402. The combined reservoir 402 and cap 420 enclose the internal components and maintain alignment of the electrode 408, spring assemblies 412, 414 and delivery medium during operation. The ridged design of the cap 420 provides mechanical stability and sealing, maintaining alignment and preventing displacement of the buffer gel, RNA pad, and electrode during use.

During iontophoretic delivery, the RNA pad 404 and the buffer gel 406 can undergo dimensional changes, including swelling or contraction due to variable hydration levels and consumption or depletion of RNA molecules and excipients. Such volumetric changes can alter the geometry and contact forces within the device and potentially lead to inconsistent ionic conduction or reduced efficiency of drug delivery. To compensate, the device and system 400 incorporate a spring-loaded assembly 412 designed to maintain the structural and functional stability of the iontophoretic therapeutic RNA delivery device during operation.

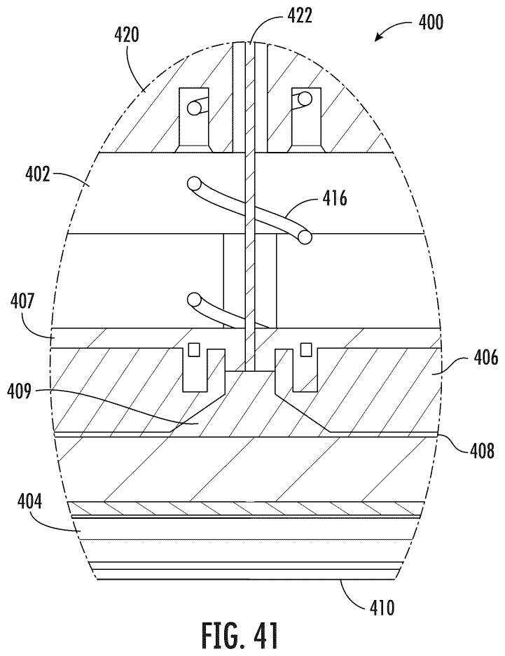

As shown in FIGS. 40 and 41, the spring-loaded assembly 412 is positioned at the top of the reservoir 402 for applying a constant biasing force for maintaining the buffer gel, RNA pad, and associated components under constant and uniform compression. The spring-loaded assembly 412 comprises a compression spring 416 having an upper end and a lower end. The upper end of the spring 416 fits over a post projecting inwardly from the cap 420. The inner end of the spring 416 fits against a rigid plastic buffer gel substrate 407. The buffer gel substrate 407 spans the interior of the reservoir 402 and is longitudinally slidable along the reservoir 402. This arrangement applies a continuous biasing force and in the direction of the membrane 410 thereby sandwiching the internal components, including the RNA pad 404 and the buffer gel 406 together under constant compression despite changes in hydration or depletion. This configuration also ensures continuous physical contact of the entire electrode surface 408 into the gel 406 which maximizes current carrying capacity. This leads to consistent electrical conduction and function of the device. The spring 416 also forces the components of the device 400 against the target tissue and stable iontophoretic performance throughout a course of iontophoretic treatment. This configuration also ensures continuous physical contact of the device 400 against the target tissue and stable iontophoretic performance throughout a course of iontophoretic treatment. By maintaining constant compression, the assembly promotes uniform ionic transport and drug delivery performance, reliable dosing of the RNA molecules, and reproducible therapeutic outcomes. A second, bottom spring assembly (not shown) may also be provided for further enhancing the compression force.

A fixation skirt 418 is adhesively secured to the RNA delivery device 400. The fixation skirt 418 facilitates temporary surgical attachment of the device to the target tissue at the treatment site. The fixation skirt may be formed of a porous mesh or textile material. The preferred adhesive provides stable coupling between the fixation skirt and the device body, ensuring that the skirt can function as a structural anchor during placement and use. This attachment is critical for maintaining device 400 positioning relative to the target tissue under treatment. The fixation skirt 418 can be sutured or stapled into place onto the target tissue, thereby stabilizing the delivery apparatus during iontophoretic operation and ensuring reliable positioning and consistent contact with the tissue.

In use, the RNA delivery device 400 is secured to a site of target tissue via the fixation skirt 418 anchored between the device and the tissue. The RNA pad 404 is loaded with an aqueous RNA solution and positioned within the slotted reservoir 402 adjacent to the buffer gel 406. The spring-loaded assembly 412 compresses the components together to maintain stable contact. The platinum electrode 408 applies electrical current across the reservoir 402, driving iontophoretic transport of RNA molecules across the membrane 410 and into the target tissue. The buffer gel 406 stabilizes and maintains local pH during iontophoretic delivery. The RNA pad 404 provides a sustained source of RNA while the semipermeable membrane 410 delivers the RNA to the target tissue. The cooperative interaction of these components enables efficient, controlled, and reproducible delivery of RNA molecules into the target tissue, while preserving molecular integrity of the RNA molecules and ensuring consistent therapeutic outcomes.

The reservoir assemblies as described and shown herein have many advantages, including use in systems and methods for drug delivery using iontophoresis. The design of each reservoir assembly minimizes gas bubble formation and adherence of the bubbles to the surface of the electrode. Gas bubbles that do form are swept away by fluid flow through the reservoir and do not collect on the electrode providing stable, consistent performance. The reservoir assemblies and iontophoresis systems and methods can be used to treat solid tumors such as pancreatic tumors as well as, but not limited to, sarcomas, head and neck, and breast cancer, both internally and externally.

Although the present invention has been shown and described in considerable detail with respect to only a few exemplary embodiments thereof, it should be understood by those skilled in the art that we do not intend to limit the reservoir assemblies to the embodiments since various modifications, omissions and additions may be made to the disclosed embodiments without materially departing from the novel teachings and advantages, particularly in light of the foregoing teachings. Accordingly, we intend to cover all such modifications, omission, additions and equivalents as may be included within the spirit and scope of the reservoir assembly as defined by the following claims. In the claims, means-plus-function clauses are intended to cover the structures described herein as performing the recited function and not only structural equivalents but also equivalent structures. Thus, although a nail and a screw may not be structural equivalents in that a nail employs a cylindrical surface to secure wooden parts together, whereas a screw employs a helical surface, in the environment of fastening wooden parts, a nail and a screw may be equivalent structures.

We claim:

1. A surgically implantable reservoir for implantation into a patient for use in an iontophoresis system for local drug delivery through a target site of internal body tissue, the iontophoresis system including a source electrode and a counter electrode in electrical communication with the source electrode for forming a localized electric field at the target site, the reservoir comprising:
a housing having an inner surface defining
a first enclosed chamber having an inlet opening spaced from an outlet opening for flow of fluid including the drug through the first chamber from the inlet opening to the outlet opening, and
a second enclosed chamber having a smaller volume than the first chamber and an inlet opening spaced from an outlet opening for flow of fluid including the drug through the second chamber from the inlet opening to the outlet opening, wherein outlet of the first chamber is in fluid communication with the inlet of the second chamber, and wherein the second chamber is adapted for holding the source electrode,
the housing comprising
a first membrane at least partially defining the first chamber and capable of interacting with the localized electric field, the membrane allowing drug to pass through the membrane and into the targeted tissue when the localized electric field is applied, and
a second membrane separating the first chamber from the second chamber,, the second membrane allowing drug to pass through the membrane and into the first chamber when the localized electric field is applied; and
means for securing the housing to the tissue of the target site,
wherein fluid flow through the second chamber from the inlet opening to the outlet opening moves gas bubbles formed by electrolysis from the surface of the electrode and carries the bubbles through the outlet opening of the second chamber.

2. The reservoir as recited in claim 1, wherein the outlet opening in the second chamber is opposite the inlet opening of the second chamber.

3. The reservoir as recited in claim 1, wherein the housing securing means comprises a skirt around at least a portion of the reservoir, wherein the skirt may be sutured to tissue at the target site.

4. The reservoir as recited in claim 3, wherein the skirt has suture openings.

5. The reservoir as recited in claim 1, wherein the housing securing means comprises a biological adhesive, microneedles, or staples.

6. The reservoir as recited in claim 1, wherein the membrane comprises natural or synthetic polyomers, including cellulose acetate, polysulfone, polycarbonate, polyamide, and polyacryl-polyamide acrylate.

7. A reservoir for use in an iontophoresis system for local drug delivery through an external target site of body tissue, the iontophoresis system including a source electrode and a counter electrode in electrical communication with the source electrode for forming a localized electric field at the target site, the reservoir comprising:
a housing for accommodating the source electrode, the housing having an inner surface defining an enclosed chamber having an inlet opening spaced from an outlet opening for flow of fluid including the drug through the chamber from the inlet opening to the outlet opening,
a membrane at least partially defining the chamber and capable of interacting with the localized electric field, the membrane allowing drug to pass through the membrane and into the targeted tissue when the localized electric field is applied, an inlet tube in fluid communication with the inlet of the housing;
an outlet tube in fluid communication with the outlet of the housing and extending at an angle of between about 30 degrees and about 90 degrees relative to a plane including the membrane;
and
a handle secured to the housing for manual placement of the membrane against the tissue of the target site,
wherein fluid flow through the chamber from the inlet opening to the outlet opening moves gas bubbles formed by electrolysis from the surface of the electrode and carries the bubbles through the outlet opening.

8. The reservoir as recited in claim 1, wherein the outlet opening is opposite the inlet opening.

9. The reservoir as recited in claim 1, wherein the membrane comprises natural or synthetic polyomers, including cellulose acetate, polysulfone, polycarbonate, polyamide, and polyacryl-polyamide acrylate.

10. An iontophoresis system for local drug delivery through a target site of internal body tissue, the iontophoresis system comprising:
a source electrode;
a counter electrode in electrical communication with the source electrode, the counter electrode being configured to cooperate with the source electrode to form a localized electric field at the target site;
a fluid cargo including the drug, the fluid cargo capable of being delivered through the tissue of the target site when exposed to the localized electric field formed between the source electrode and the counter electrode;
a surgically implantable reservoir adapted to be secured to the target site, the reservoir comprising
a housing having an inner surface defining an enclosed chamber and an inlet opening and an outlet opening for cargo flow through the chamber, the housing capable of interacting with the localized electric field to release the cargo,
a fluid channel from the outlet opening to the exterior of the housing, and
a platform extending inwardly into the chamber from the inner surface of the housing such that the platform and an adjacent portion of the inner surface of the housing define a trough surrounding the platform, the platform configured for holding the source electrode; and
means for securing the housing to the tissue of the target site,
wherein cargo flow through the reservoir from the inlet opening to the outlet opening moves gas bubbles formed by electrolysis from the surface of the electrode and carries the bubbles through the outlet opening and not the channel exterior of the housing.

11. The iontophoresis system as recited in claim 10, wherein the source electrode comprises platinum.

12. The iontophoresis system as recited in claim 10, wherein the cargo comprises anesthetics, vaccines, chemotherapeutic agents, metabotites, immunomodutators, antioxidants, antibiotics, and ion channel regulators, or hormones.

13. The iontophoresis system as recited in claim 12, wherein the cargo further comprises one or more pharmaceutically acceptable carriers, excipients, or diluents.

14. The iontophoresis system as recited in claim 10, wherein the cargo comprises a therapeutic agent.

15. The iontophoresis system as recited in claim 14, wherein the therapeutic agent comprises gemcitabine.

16. The iontophoresis system as recited in claim 10, further comprising a source of cargo in fluid communication with the inlet opening for a flow of cargo into the housing.

17. The iontophoresis system as recited in claim 10, wherein the outlet opening is spaced from the inlet opening.

18. The iontophoresis system as recited in claim 17, wherein the outlet opening is opposite the inlet opening.

19. The iontophoresis system as recited in claim 10, wherein the housing securing means comprises a skirt around at least a portion of the reservoir, wherein the skirt may be sutured to tissue at the target site.

20. The iontophoresis system as recited in claim 19, wherein the skirt comprises a plurality of anchor points defining suture openings.

20. The iontophoresis system as recited in claim 10, wherein the housing securing means comprises a biological adhesive.

22. The iontophoresis system as recited in claim 10, wherein at least a portion of the housing comprises a membrane, the membrane allowing drug to pass through the membrane and into the targeted tissue when a localized electric field is applied.

23. The iontophoresis system as recited in claim 22, wherein the membrane comprises natural or synthetic polyomers, including cellulose acetate, polysulfone, polycarbonate, polyamide, or polyacryl-polyamide acrylate.

24. A surgically implantable reservoir for implantation into a patient for use in an ontophoresis system for local delivery of ribonucleic acid molecules through a target site of internal body tissue, the iontophoresis system including a source electrode and a counter electrode in electrical communication with the source electrode for forming a localized electric field at the target site, the reservoir comprising:
a housing having an inner surface defining an interior cavity open one side for accommodating the electrode;
a membrane spanning the open side of the housing for covering the cavity in the housing forming an enclosed chamber, the membrane capable of interacting with the localized electric field for allowing RNA molecules to pass through the membrane and into the targeted tissue when the localized electric field is applied;
an absorbent pad for holding the ribonucleic acid molecules and disposed within the chamber;
a buffer gel partially filling the chamber;
a spring assembly for biasing the electrode into the buffer gel and toward the pad and the membrane; and
means for securing the housing to the tissue of the target site.

25. The iontophoresis system as recited in claim 24, wherein the housing securing means comprises a skirt around at least a portion of the reservoir, wherein the skirt may be sutured to tissue at the target site.

26. The iontophoresis system as recited in claim 24, wherein the skirt comprises a plurality of anchor points defining suture openings.

27. The iontophoresis system as recited in claim 24, wherein the housing securing means comprises a biological adhesive.

28. The iontophoresis system as recited in claim 24, wherein the membrane comprises natural or synthetic polymers, including cellulose acetate, polysulfone, polycarbonate, polyamide, or polyacryl-polyamide acrylate.
